(19) United States
(12) Patent Application Publication
(10) Pub. No.: US 2018/0185167 A1
(43) Pub. Date: Jul. 5, 2018

(54) METHODS AND SYSTEMS TO DETERMINE PROPER SEATING OF A FIRST ORTHOPEDIC COMPONENT WITH A SECOND ORTHOPEDIC COMPONENT

(71) Applicants: Trey Rodgers, Warsaw, IN (US); Jordan H. Freedman, Fort Wayne, IN (US)

(72) Inventors: Trey Rodgers, Warsaw, IN (US); Jordan H. Freedman, Fort Wayne, IN (US)

(21) Appl. No.: 15/856,970

(22) Filed: Dec. 28, 2017

Related U.S. Application Data
(60) Provisional application No. 62/440,715, filed on Dec. 30, 2016.

(51) Int. Cl. A61F 2/46

(52) U.S. Cl. CPC A61F 2/468; A61F 2/3609

(57) ABSTRACT

Disclosed are methods and systems to determine proper seating engagement of a first orthopedic component with a second orthopedic component to ensure stability of the assembled implant. In an example, the first orthopedic component is a head for a shoulder or hip prosthesis and the second orthopedic component is a stem for the shoulder or hip prosthesis, and the head and stem can be attached via a taper junction. The methods and systems can include contacting the first or second orthopedic component to excite the first or second orthopedic component and generate a measurable response, and evaluating the measured response to determine whether the first orthopedic component is properly seated with the second orthopedic component. In an example, a tooling component can physically contact the first or second orthopedic component and generate a vibrational response. The same tooling component or a second tooling component can include one or more sensors to measure the vibrational response and provide an output to the user.

### PRIORITY CLAIM

This application claims the benefit of priority of U.S. Provisional Patent Application Ser. No. 62/440,715, filed on Dec. 30, 2016, and which is herein incorporated by reference in its entirety.

### TECHNICAL FIELD

The present application relates to orthopedics, and more particularly, to systems and methods to ensure proper seating engagement at a tapered junction between a first orthopedic component and a second orthopedic component.

### BACKGROUND

Orthopedic implants may be used for the replacement of all, or a portion of, a patient's joint. For example, total hip arthroplasty may be used to restore function to a diseased or injured hip joint. Similarly, total shoulder replacement may be used to restore function to an arthritic shoulder. The hip and the shoulder are both examples of ball and socket joints in which one bone articulates relative to the other bone. In the case of the hip joint, the femoral head articulates relative to the hip socket formed by the acetabulum. In total hip arthroplasty, the hip implant can include a femoral head that attaches to a femoral stem implanted in the femoral canal of a patient.

In some hip implant designs, a taper junction can be used between the femoral head and stem for fixation strength of the head with the stem. The femoral head is typically placed on a proximal end of the stem and then the head is impacted by physical force such that the femoral head travels down at least a portion of the taper of the stem.

Problems can ensue if the head is not properly seated with the taper of the stem. Trunnionosis refers to implant wearing at the taper junction, which can lead to implant failure, including fretting and corrosion. Research suggests that a contributing factor to trunnionosis is the impaction of the femoral head during seating. Instruments can be used during impaction to measure the impaction force. However, force alone may not be indicative of proper seating, since impaction can be operator dependent and can be affected by more than just force. For example, sufficient force could be applied, but the head may not be oriented at a proper angle relative to the neck of the stem.

### Overview

The present inventors recognize, among other things, an opportunity for improved methods and systems to determine proper seating engagement of a prosthetic head with a corresponding prosthetic stem, following impaction of the head onto the tapered proximal end of the stem. In an example, the head can be a femoral head for a hip implant and the stem can be a femoral stem for implantation in the femur. Various types of devices or tooling can be used to contact at least one of the head and stem to excite the head or stem and generate a measurable response. Such response can include, for example, an acoustic response or a vibrational response. If the head is properly seated with the stem, the resulting response will be at steady state and a consistent and repeating wave can be generated.

To further illustrate the systems and methods disclosed herein, a non-limiting list of examples is provided here:

Examples according to the present application can include a method of determining proper seating engagement of a first component with a second component. The first component can include a recess with a taper configured to at least partially receive a tapered portion at an end of the second component. The method can include contacting at least one of the first and second components to excite the at least one of the first and second components and generate a measurable response, measuring the response generated by the at least one of the first and second components, and evaluating whether the measured response equates to proper seating engagement of the first component with the second component. In an example, the measured response can include a steady state response observed over a period of time and such steady state response can equate to proper seating engagement. In another example, proper seating engagement can be achieved when the measured response is approximately equal to a predetermined value. In an example, the first component can be a femoral head and the second component can be a femoral stem.

Examples according to the present application can include a method of securing an orthopedic head on a stem having a tapered proximal portion. The method can include placing the head on the stem, the head having an interior recess with a taper corresponding to the tapered proximal portion of the stem. The method can further include impacting the head to secure the head on the stem, the head moving relative to the tapered proximal portion of the stem during impaction. The method can further include contacting the head or the stem to generate an energy response, and measuring the energy response to determine if the head is in proper seating engagement with the tapered proximal portion of the stem. In an example, contacting the head or the stem can include physically contacting the head or the stem with a device that causes a vibrational response. In an example, measuring the energy response can include releasably engaging a measurement tool with the head or the stem. In an example, the measurement tool can include an accelerometer. In an example, the head can be a femoral head of a hip prosthesis and the stem can be a femoral stem for implantation in a femur.

Examples according to the present application can include a system for ensuring proper seating engagement of an orthopedic head with a stem having a tapered proximal portion. The system can comprise a first tooling component for contacting the head or the stem to excite the head or the stem and generate a measurable response, and a second tooling component for measuring the response generated by the head or the stem after the first tooling component contacts the head or the stem. The first tooling component can be an impaction tool for impacting the head on the stem to secure the head with the stem or the first tooling component can be separate from the impaction tool and excite the head or stem after impaction of the head on the stem. The system can comprise one or more sensors for measuring a dynamic change of the head or stem after the first component excites the head or stem. The system can comprise an electronic component configured to receive an electrical signal from one or more sensors and output on a display one or more values correlating to the measured response. In an example, the stem or head can be designed to include one or more features configured to increase a vibrational sensitivity of the stem or head.

These and other examples and features of the present systems and methods will be set forth in part in the following Detailed Description. This Overview is intended to provide non-limiting examples of the present subject matter—it is not intended to provide an exclusive or exhaustive explanation. The Detailed Description below is included to provide further information about the present systems and methods.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a perspective view of an example hip prosthesis including a femoral head and stem, as well as an acetabular cup.

FIG. 2 shows the femoral head and stem of FIG. 1 implanted in a femur of a patient.

FIG. 3 is a cross-sectional view of the femoral head and an end portion of the stem to illustrate a taper fit between the head and stem.

FIG. 4A is a cross-sectional view of an example tapered end portion of the stem.

FIG. 4B is a cross-sectional view of the tapered end portion of the stem of FIG. 4A.

FIG. 5 shows the implanted femoral head and stem of FIG. 1 and an example measurement tool removably engaged with the stem.

FIG. 6 is a partial cross-sectional view of the femoral head, stem and measurement tool of FIG. 5.

FIG. 7 shows the implanted femoral head and stem of FIG. 1 and an example measurement tool removably engaged with the cup.

FIG. 8 shows the implanted femoral head and measurement tool of FIG. 7, as well as an example contact tool and an example display system.

### DETAILED DESCRIPTION

The present application relates to systems and methods for use with a prosthesis having a taper junction to determine if a first component of the prosthesis is properly seated with a second component of the prosthesis. The first component can include a female tapered bore and the second component can include a corresponding male tapered trunnion. In an example, the first component can be a femoral head of a hip prosthesis and the second component can be a femoral stem of the hip prosthesis. In another example, the prosthesis can be a shoulder prosthesis. The present application includes a method of determining proper seating engagement through contacting the first or second component to excite the component and generate a dynamic response, and measuring the response generated to evaluate whether the response equates to proper seating engagement. For purposes herein, “proper seating” or “proper seating engagement” refers to attachment of the first component to the second component such that the attachment strength between the components is high and the first component is stable on the second component. As a result of proper seating engagement, micro-motion can be eliminated or reduced. As described below, the femoral head and a trunnion on the stem are designed for high compressive strength when the head is in proper seated engagement with the trunnion, due in part to the taper junction between the head and trunnion.

The present application can include a system for determining proper seating engagement including a first tooling component for exciting the first or second component and a second tooling component for measuring a response generated by the first or second component. In an example, the first tooling component can physically contact one or both of the head and stem. In another example, the first tooling component can use other means to excite one or both of the head and stem without the first tooling component contacting the head or stem directly. The system can include a second tooling component for measuring a response generated by the head or stem after excitation. The second tooling component can include a sensor configured to generate an output usable by the surgeon or other user of the system. The first and second tooling components can be part of the same tooling assembly or be separate from one another. The above features are described in further detail below.

FIG. 1 shows an example hip prosthesis 10 which can include an acetabular shell 12, acetabular liner 14, femoral head 16, and stem 18. The acetabular shell 12 and liner 14 can be configured for implantation in the acetabulum. The femoral stem 18 can be configured for implantation in a femur of a patient and the femoral head 16 can articulate relative to the acetabular liner 14. The stem 18 can include a distal end 20 and a proximal end 22. A neck 24 can be located at the proximal end 22 and can include a tapered portion, which is described below.

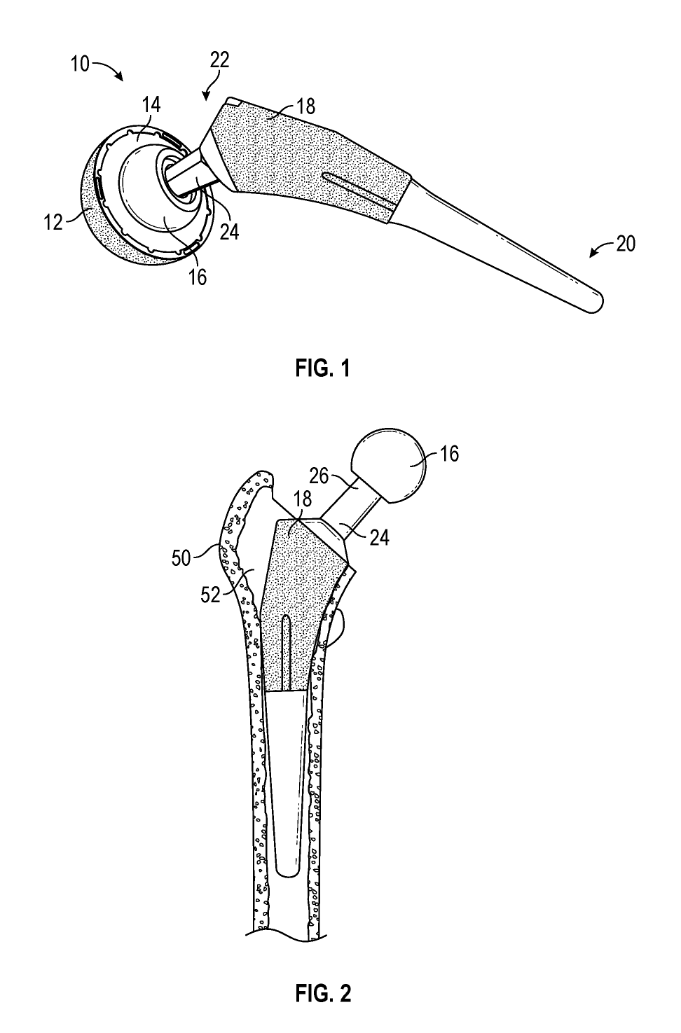

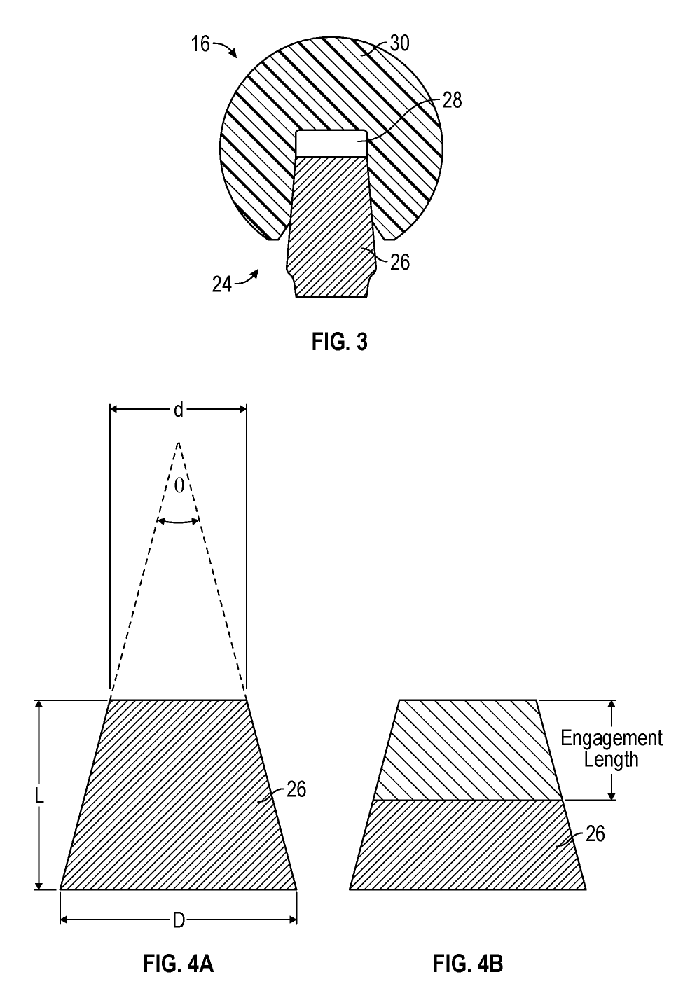

FIG. 2 shows a femur 50 of a patient, as well as the femoral head 16 and stem 18 of FIG. 1 after the stem 18 has been implanted in a canal 52 of the femur 50. (The stem 18 and head 16 can be referred to herein collectively as a construct.) For simplicity, FIG. 2 shows the femur 50 only and not the pelvis, which would include the acetabular shell 12 and liner 14 of FIG. 1 implanted therein at a conclusion of the hip arthroplasty. During surgery, typically the stem 18 can be implanted into the canal 52 and then the femoral head 16 can be impacted onto the neck 24 of the stem 18. Specifically, the head 16 can be impacted onto a trunnion 26 of the neck 24 which can be tapered, as described below and shown in FIGS. 3, 4A and 4B.

FIG. 3 is a cross-sectional view of the femoral head 16 and the neck 24 of FIG. 2 with the femoral head shown at least partially seated with the taper of the trunnion 26. The head 16 can include a tapered recess or bore 28 that can extend through a portion of an interior 30 of the head 16. The tapered bore 28 and the tapered trunnion 26 can be configured such that when the head 16 is placed on the neck 24 and a force is imparted on the head 16, the head 16 and the trunnion 26 can be forced together through mechanical interlocking, resulting in a stable fixation. Note that the head 16 and trunnion 26 are oriented in FIG. 3 with the bore 28 and trunnion 26 oriented upright, although as shown in FIGS. 1 and 2, the bore 28 and trunnion 26 are oriented at an angle relative to a length of the stem 18 and relative to a horizontal plane, when the stem 16 is implanted in the femur. In an example, after proper seating, a headspace or gap can be present between the trunnion 26 and the interior 30 of the head 16, as shown in FIG. 3.

FIGS. 4A and 4B are cross-sectional views of the trunnion 26 of FIG. 3 and are included herein to note features of the trunnion 26 and the taper junction design. Such features can include taper angle θ, taper length L and engagement length EL. In some designs, the trunnion 26 can be designed to have a shallow taper angle θ and a reduced taper length L. In an example, the taper angle θ can range between about 3 and about 8 degrees if the angle is measured as a half angle (i.e. the angle is measured from one face of the trunnion 26 to the centerline). It is recognized that the angle would be doubled if the angle is measured from one taper face to the other taper face. Although the range of about 3 to about 8 degrees is provided above for the taper angle θ, it is recognized that the taper angle θ of the trunnion 26 can be more of less than the provided range. The engagement length EL can be defined as a length of the trunnion 26 that is engaged with the bore 28 of the head 16 after the head 16 has been fully seated with the stem 18 (i.e. achieved proper seating engagement). It is recognized that the head 16 may not travel far on the trunnion 26 and the engagement length EL can be a relatively small number; however, it can be important to the success of the construct and its lifespan that, during impaction, the head 16 travel the engagement length EL such that the head 16 and the trunnion 26 are fully engaged.

Given the design of the bore 28 and trunnion 26, the seated construct can have a high compressive strength. However, it can be difficult to measure the strength of such attachment directly. Consequently, as provided above in the Background section, techniques and systems can be used to measure the impaction force of the head onto the stem during impaction; however, this is an indirect measurement technique to determine proper seating engagement and does not necessarily correlate to proper seating (i.e. sufficient or optimal attachment strength). Other factors, such as, for example, angle, can impact the strength of the seated construct. For example, if the head 16 is not properly placed on the stem 18 such that the bore 28 of the head 16 is aligned with the taper of the trunnion 26, the head 16 may not be properly seated with the stem 18.

Described below are examples of tooling assemblies and methods for directly determining whether the head 16 is properly seated with the stem 18. Such tooling assemblies and methods of the present application can be used during and/or after impaction of the head 16 onto the stem 18. Such tooling assemblies and methods of the present application can be used for at least one of exciting the construct and measuring a dynamic response resulting from the excitation. Such tooling assemblies can include one or more tooling components that can perform these functions through physical contact with the construct or using other methods to excite the construct and measure the response. The measured response can be used to determine if the head is properly seated.

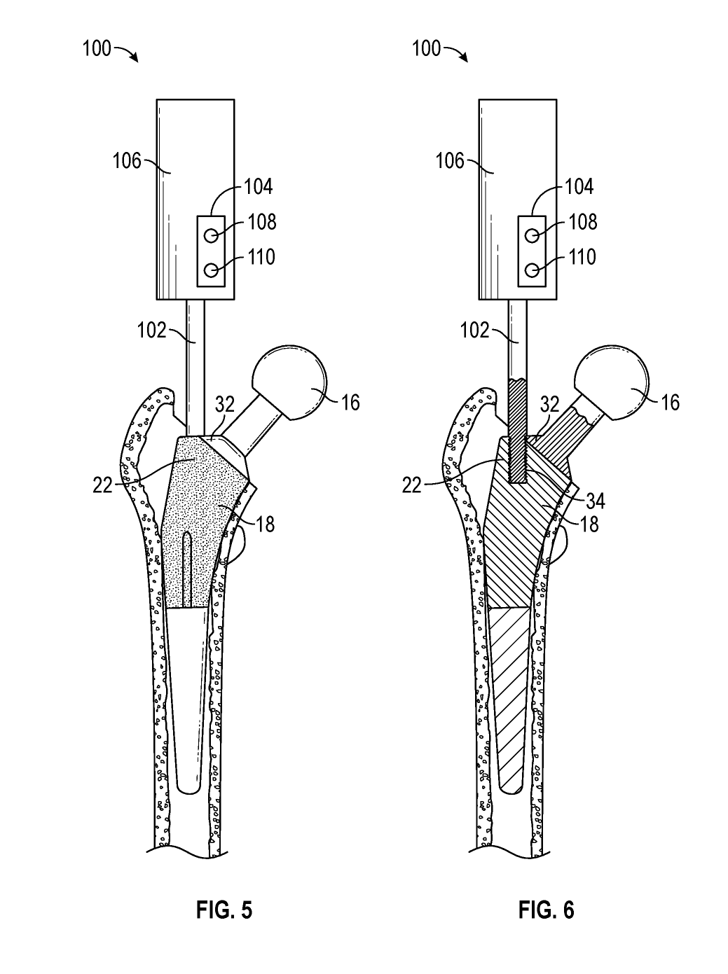

FIG. 5 shows the implanted stem 18 and head 16 of FIG. 4, as well as an example tooling component 100 for physically contacting a region 32 of the stem 18 in proximity to the proximal end 22. In an example, the tooling component 100 can be configured to removably engage with the region 32 of the stem 18 such that the tooling component 100 can be securely attached to the stem 18 temporarily. Various types of engagement can be used to temporarily secure the tooling component 100 to the stem 18. FIG. 6 shows one example of such engagement. FIG. 6 illustrates the tooling component 100, stem 18 and head 16 from FIG. 5, with a portion of the stem 18 and tooling component 100 displayed in cross-section. As shown in FIG. 6, the tooling component 100 can have a threaded engagement with the stem 18. The stem 18 can include a threaded bore 34 configured to receive a threaded portion of shaft 102 of the tooling component 100.

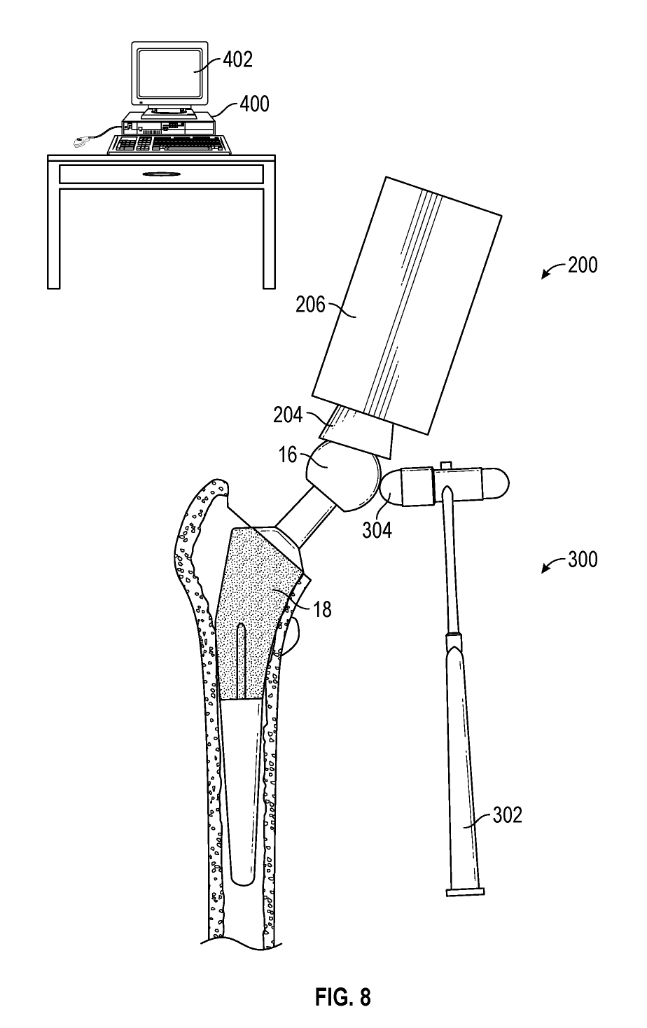

In an example, the tooling component 100 can be configured to measure a vibrational profile of the stem 18 in the region 32. The tooling component 100 or another (second) tooling component not shown in FIGS. 5 and 6 can be configured to excite the stem 18 in an area where the stem 18 is exposed (i.e. not within the canal 52). (An example of such excitation is described below and shown in FIG. 8.) As a result of such excitation, the stem 18 can produce a vibrational response in the form of a measurable wave. If the head 16 is not properly seated with the stem 18, the generated wave can change in amplitude and/or frequency over time because the head 16 is not stable on the stem 18. In contrast, if the generated wave is a repeating, consistent wave of equal amplitude and frequency over time, this can equate to proper seating engagement of the head 16 with the stem 18. The repeating, consistent wave can be indicative of a steady state vibrational response that would not occur if the head 16 was not stable on the stem 18.

As an alternative to or in addition to evaluating whether the vibrational response is at steady state, the methods and systems of the present application can include monitoring the vibrational response for a specific value equating to proper seating engagement. This may require knowledge, prior to impaction, of the specific value for that construct that corresponds with proper seating. Such value can depend, at least in part, on the material and geometry of the particular construct and the particular excitation to be used. In an example, it may require seating the head 16 on the stem 18 before the stem 18 is implanted in the patient to determine this value using the same type and quantity of excitation that will be used when the stem 18 is implanted in the femur. Alternatively, a model can be used to determine the value based on, at least in part, the specifications of the stem 18 and head 16 and the type and quantity of excitation to be used to generate the vibrational response. One advantage of monitoring for a steady state response rather than a particular value is that the specific amplitude does not have to be determined prior to impact. A vibrational response profile or a pattern of a steady state waveform can be observed rather than observing whether a specific value is achieved. The response profile can include the amplitude of the waveform.

The tooling component 100 can include one or more sensors configured to sense and measure the vibration of the stem 18. In an example, the one or more sensors can include an accelerometer, such as a piezoelectric accelerometer. The accelerometer can output a signal (for example, an electrical signal) in response to the vibrational changes measured by the accelerometer. The output of the one or more sensors is described further below. In an example, the tooling component 100 can include a visual output 104 on an exterior of a body 106 of the tooling component 100. The visual output 104 can include a first light 108 and a second light 110. In an example, the first light 108 can be a first color (for example, red) indicative that proper seating engagement is not achieved, and the second light 110 can be a second color (for example, green) indicative of proper seating engagement.

FIGS. 5 and 6 illustrate one example of a location on the construct where the tooling component 100 can contact the construct. It is recognized that the tooling component 100 (or similar tooling) can be configured to contact other regions of the stem 18 or head 16 that are exposed (i.e. not implanted within the canal 52). As provided above, the tooling component can have a different type of engagement as compared to the threaded engagement of FIG. 6. An advantage of a threaded engagement is that the tooling component 100 becomes part of the construct and can be used to amplify a vibrational response of the construct.

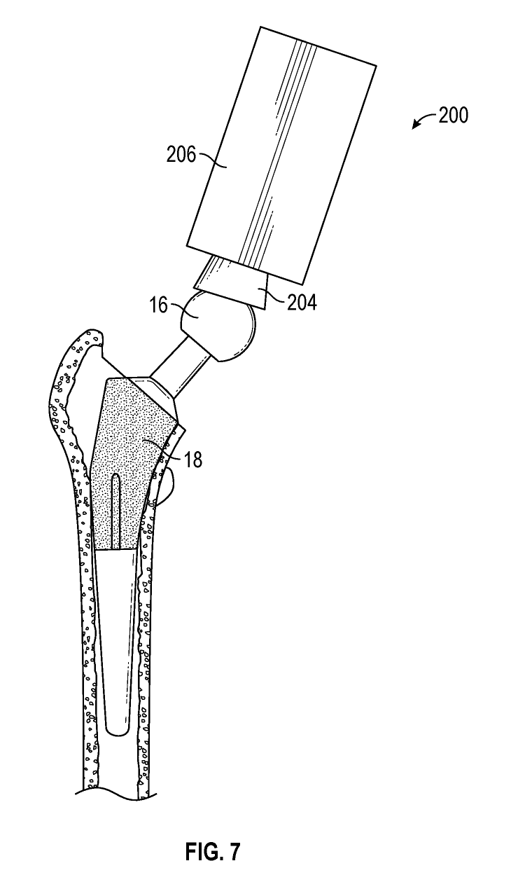

FIG. 7 illustrates an example tooling component 200 that can be configured for contact with the head 16 rather than the stem 18. The tooling component 200 can include a feature 204 configured to engage with a top portion of the head 16. As similarly described above for the tooling component 100, the tooling component 200 can be configured to measure a vibrational response of the head 16 after the head 16 is excited.

The feature 204 can encompass various types of engagement designs for releasably engaging the tooling component 200 with the head 16. In an example, the feature 204 can include a threaded component configured to engage with a threaded bore in the head, similar to the design illustrated in FIG. 6. In an example, the feature 204 can include a protrusion configured to engage with a groove formed in an exterior surface of the head 16.

In addition to their measurement capabilities, either or both of the tooling components 100 and 200 can include an additional feature (not specifically shown in FIGS. 5-7) for exciting the stem 18 and/or head 16. In an example, such excitation or contact feature can be housed within the body 106 of tooling component 100 or a body 206 of tooling component 200. In an example, the feature can be moved to an active position in which some or all of the feature is outside of the body 106 or 206 and can physically contact the stem 18 and/or head 16.

FIG. 8 shows the tooling component 200 of FIG. 7 attached to the head 16 and additionally shows a second tooling component 300 configured to physically contact the head 16 to excite the head 16. In an example, the second tooling component 300 can be a rubber mallet configured to ring the head 16. A user can grip a handle portion 302 of the component 300 to move the tooling component 300 such that a contact portion 304 of the component can exert a force on the head 16 to ring the head 16. Such ringing or exciting of the head 16 can be similar to the manner in which a tuning fork can be rung and can generate a vibrational response caused by physical movement of the tuning fork/head 16. As provided above, an output of the vibrational response can include a measurable wave that can be plotted as a function of time.

In an example, the second tooling component 300 can contact a side portion of the head 16 (as shown in FIG. 8) to ring the head 16, after the head 16 is impacted onto the stem 18. In another example, the second tooling component can be an impaction tool configured to exert a force on the head 16 during a typical impaction step in the implantation of the hip prosthesis. (In such an example, the tooling component 200 may contact a side portion of the head 16 and the impaction tool may contact a top region of the head 16.) The tooling component 200 can measure the vibrational response of the head 16 during impaction. As described above in reference to FIG. 5, the tooling component 200 can be configured to measure the response to determine whether the response is at steady state and/or whether a specific predetermined value is achieved. The results of the response can be communicated to the user directly from the tooling component 200 and/or through electrical equipment. The determination of proper seating of the head 16 with the stem 18 can occur during or after impaction.

The methods and systems of the present application can include sensing and measuring the dynamic change in the stem 18 and/or head 16 in response to the stem 18 and/or head 16 being excited as described above. In an example, the tooling components 100 and 200 can each include one or more sensors configured to measure the dynamic change of the stem 18 and/or head 16 caused by such excitation. The one or more sensors can generate an output that can be communicated directly to the user or sent to a device inside or outside the surgical room.

In an example, the output can be in the form of audio or visual feedback to the user. For example, either or both of the tooling components 100 and 200 can include one or more sensors for measuring the dynamic change (such as a vibrational change) of the stem 18 and/or head 16. If the measured response equates to proper seating engagement, the tooling components 100 and 200 can be configured to provide the user with a sound, such as a beep, or a visual cue, such as a green light, to indicate proper seating engagement. (See, for example, the visual output 104 in FIGS. 5 and 6, which can include first light 108 and second light 110.) In that case, the feedback to the user can be part of the design of the tooling components 100 and 200.

In an example, the data collected by the one or more sensors can be sent (via a wired or wireless connection) to an electrical device inside or outside the surgical room, which can display the data from the one or more sensors and/or output a positive or negative indicator of proper seating engagement. FIG. 8 shows an example electrical component for use with the tooling components 200 and 300—a computer 400 can be configured for communication with the one or more sensors such that the computer 400 can receive the data from the one or more sensors and display the data on a display 402. The display 402 can show some or all of the data from the sensors and/or show a conclusion (affirmative or negative) as to whether proper seating engagement is achieved. The measuring and data collection can be continuous or semi-continuous during impaction and/or excitation of the stem 18 and/or head 16.

The figures included herein illustrates examples of tooling components that can be used and how such tooling components can interact with the head 16 and/or stem 18 to determine proper seating. Various types and designs of tooling components can be used in the methods and systems of the present application. In an example, the tooling component can include a clip configured to temporarily attach to the stem 18 and/or head 16. Various features can be included in the design of the tooling component to alert the user once the tooling component has engaged with the stem 18 and/or head 16. In an example, a magnetic engagement can be used between the tooling component and the stem 18 and/or head 16.

The methods and systems of the present application include exciting the stem 18 and/or head 16 to generate a measurable response. The above description focuses on measuring a vibrational response of the stem and/or head 16. It is recognized that other types of dynamic changes to the stem 18 and/or head 16 can be measured. In an example, the stem 18 and/or head 16 can be designed to have an acoustic profile such that the stem 18 and/or head 16 generates an acoustic response when the stem 18 and/or head 16 is excited. It is recognized that the stem 18 and/or head 16 can be excited (and thus generate a measurable response) in a number of ways. Although physical contact of the tooling components with the stem 18 and/or head 16 is focused on herein, it is recognized that non-physical contact of the tooling component with the stem 18 and/or head 16 can be used to excite the stem 18 and/or head 16. Such examples can include contacting the stem 18 and/or head 16 using ultrasound, lasers, reflected waves, etc.

In an example, the design of the stem 18 and/or head 16 can be used to increase a sensitivity of the stem 18 and/or head 16 to excitation and thus increase a vibrational or acoustic response of the construct. This can be accomplished, for example, through material selection, geometrical design, etc. In an example in which a vibrational profile of the construct is used for measurement, the vibrational nodes of the construct can be used to increase the vibrational response. The location of one or more vibrational nodes on the construct can be controlled so that the vibrational response is measured at a peak of the wave, rather than at the node. The location of the nodes can be controlled through thickness and stiffness variability on the construct, material selection, heat treatment, etc.

The methods and systems provided herein focus on exciting the head 16 and/or stem 18 such that the head 16 and/or stem 18 can generate a measurable response. The techniques and tools used for exciting the construct can vary as provided above, as can the measurement techniques and tooling. The methods and systems of the present application can include physically touching the construct with the tooling to excite the construct or exciting the construct through non-physical contact. The response by the construct can be measured using the same tooling for exciting the construct or a second tooling component. Such measurements can be taken during impaction of the head 16 onto the stem 18 or after the head 16 has been impacted. Although implementation of the methods and systems can take many different forms, a unifying premise of the methods and systems disclosed herein is that proper seating of the head 16 with the stem 18 can be determined by directly measuring a response generated by the head 16 and/or stem 18, rather than measuring an impaction force applied to the head 16.

Although a hip prosthesis is focused on in describing the methods and systems of the present application, it is recognized that the methods and systems of determining proper seating are not limited to only a femoral head and stem. The methods and systems described herein can be used for other orthopedic components that include a taper junction, such as, for example, components of a shoulder implant. The methods and systems described herein can also be used to determine proper attachment (and sufficient fixation) of a first component to a second component in the absence of a taper junction, particularly in those applications in which it can otherwise be difficult to determine if the first component is sufficiently attached to the second component.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The present application provides for the following exemplary embodiments or examples, the numbering of which is not to be construed as designating levels of importance:

Example 1 provides a method of determining proper seating engagement of a first component with a second component, the first component having a recess with a taper configured to at least partially receive a tapered portion at an end of the second component. The method can include contacting at least one of the first and second components to excite the at least one of the first and second components and generate a measurable response, measuring the response generated by the at least one of the first and second components, and evaluating whether the measured response equates to proper seating engagement of the first component with the second component.

Example 2 provides the method of Example 1 optionally configured such that the measured response includes a steady state response observed over a period of time, after contacting the at least one of the first and second components. The steady state response can equate to proper seating engagement.

Example 3 provides the method of Example 1 optionally configured such that proper seating engagement is achieved when the measured response is approximately equal to a predetermined value.

Example 4 provides the method of any of Examples 1-3 optionally configured such that contacting at least one of the first and second components includes physically contacting at least one of the first and second components to generate a vibrational response.

Example 5 provides the method of Example 4 optionally configured such that measuring the response generated includes using an accelerometer to measure the vibrational response caused by physically contacting the at least one of the first and second components.

Example 6 provides the method of any of Examples 1-5 optionally configured such that the first component is a femoral head and the second component is a femoral stem.

Example 7 provides a method of securing an orthopedic head on a stem having a tapered proximal portion. The method can include placing the head on the stem, the head having an interior recess with a taper corresponding to the tapered proximal portion of the stem. The method can further include impacting the head to secure the head on the stem, the head moving relative to the tapered proximal portion of the stem during impaction, contacting the head or the stem to generate an energy response, and measuring the energy response to determine if the head is in proper seating engagement with the tapered proximal portion of the stem.

Example 8 provides the method of Example 7 optionally configured such that measuring the energy response includes measuring the energy response over a period of time to determine if an output of the energy response is at steady state.

Example 9 provides the method of Example 7 optionally configured such that measuring the energy response includes comparing the measured response to a predetermined value associated with proper seating engagement of the head with the stem.

Example 10 provides the method of any of Examples 7-9 optionally configured such that contacting the head or the stem includes physically contacting the head or the stem with a device that causes a vibrational response.

Example 11 provides the method of any of Examples 7-10 optionally configured such that measuring the energy response includes releasably engaging a measurement tool with the head or the stem.

Example 12 provides the method of Example 11 optionally configured such that releasably engaging a measurement tool with the head or the stem includes a threaded engagement between the measurement tool and the head or the stem.

Example 13 provides the method of Example 11 or 12 optionally configured such that the measurement tool includes an accelerometer.

Example 14 provides a system for ensuring proper seating engagement of an orthopedic head with a stem having a tapered proximal portion. The system can comprise a first tooling component for contacting the head or the stem to excite the head or the stem and generate a measurable response, and a second tooling component for measuring the response generated by the head or the stem after the first tooling component contacts the head or the stem.

Example 15 provides the system of Example 14 optionally configured such that the first tooling component is separate from an impaction tool and the first tooling component contacts one or both of the head and the stem after impaction of the head on the tapered component of the stem.

Example 16 provides the system of Example 14 optionally configured such that the first tooling component is an impaction tool for impacting the head on the stem to secure the head with the stem.

Example 17 provides the system of any of Examples 14-16 optionally configured such that the first tooling component and the second tooling component are housed on one tooling assembly.

Example 18 provides the system of any of Examples 14-17 optionally configured such that the second tooling component temporarily engages with the head or the stem to measure the response when the first tooling component contacts one or both of the head and the stem.

Example 19 provides the system of any of Examples 14-18 optionally configured such that the second tooling component includes a sensor that measures a dynamic change of the head or the stem after the first component contacts the head or the stem.

Example 20 provides the system of Example 19 optionally further comprising an electronic component configured to receive an electrical signal from the sensor and output one or more values on a display, the one or more values correlating to the measured response.

Example 21 provides the system of any of Examples 18-20 optionally configured such that the second tooling component has a threaded feature for threaded engagement with a corresponding threaded feature on the head or the stem.

Example 22 provides the system of any of Examples 14-21 optionally configured such that the first tooling component physically contacts a portion of the head or the stem and the contacted portion of the head or the stem generates a vibrational response measured by the second component.

Example 23 provides the system of Example 22 optionally configured such that the stem or the head includes one or more features configured to increase vibrational sensitivity of the stem or the head.

Example 24 provides a system or method of any one or any combination of Examples 1-23, which can be optionally configured such that all steps or elements recited are available to use or select from.

Various aspects of the disclosure have been described. These and other aspects are within the scope of the following claims.

The claimed invention is:

1. A method of determining proper seating engagement of a first component with a second component, the first component having a recess with a taper configured to at least partially receive a tapered portion at an end of the second component, the method comprising:
contacting at least one of the first and second components to excite the at least one of the first and second components and generate a measurable response;
measuring the response generated by the at least one of the first and second components; and
evaluating whether the measured response equates to proper seating engagement of the first component with the second component.

2. The method of claim 1 wherein the measured response includes a steady state response observed over a period of time, after contacting the at least one of the first and second components, and the steady state response equates to proper seating engagement.

3. The method of claim 1 wherein proper seating engagement is achieved when the measured response is approximately equal to a predetermined value.

4. The method of claim 1 wherein contacting at least one of the first and second components includes physically contacting at least one of the first and second components to generate a vibrational response.

5. The method of claim 4 wherein measuring the response generated includes using an accelerometer to measure the vibrational response caused by physically contacting the at least one of the first and second components.

6. The method of claim 1 wherein the first component is a femoral head and the second component is a femoral stem.

7. A method of securing an orthopedic head on a stem having a tapered proximal portion, the method comprising:
placing the head on the stem, the head having an interior recess with a taper corresponding to the tapered proximal portion of the stem;
impacting the head to secure the head on the stem, the head moving relative to the tapered proximal portion of the stem during impaction;
contacting the head or the stem to generate an energy response; and
measuring the energy response to determine if the head is in proper seating engagement with the tapered proximal portion of the stem.

8. The method of claim 7 wherein measuring the energy response includes measuring the energy response over a period of time to determine if an output of the energy response is at steady state.

9. The method of claim 7 wherein measuring the energy response includes comparing the measured response to a predetermined value associated with proper seating engagement of the head with the stem.

10. The method of claim 7 wherein contacting the head or the stem includes physically contacting the head or the stem with a device that causes a vibrational response.

11. The method of claim 7 wherein measuring the energy response includes releasably engaging a measurement tool with the head or the stem.

12. The method of claim 11 wherein releasably engaging a measurement tool with the head or the stem includes a threaded engagement between the measurement tool and the head or the stem.

13. The method of claim 11 wherein the measurement tool includes an accelerometer.

14. A system for ensuring proper seating engagement of an orthopedic head with a stem having a tapered proximal portion, the system comprising:
a first tooling component for contacting the head or the stem to excite the head or the stem and generate a measurable response; and
a second tooling component for measuring the response generated by the head or the stem after the first tooling component contacts the head or the stem.

15. The system of claim 14 wherein the first tooling component is separate from an impaction tool and the first tooling component contacts one or both of the head and the stem after impaction of the head on the tapered component of the stem.

16. The system of claim 14 wherein the first tooling component is an impaction tool for impacting the head on the stem to secure the head with the stem.

17. The system of claim 14 wherein the first tooling component and the second tooling component are housed on one tooling assembly.

18. The system of claim 14 wherein the second tooling component temporarily engages with the head or the stem to measure the response when the first tooling component contacts one or both of the head and the stem.

19. The system of claim 18 wherein the second tooling component includes a sensor that measures a dynamic change of the head or the stem after the first component contacts the head or the stem.

20. The system of claim 19 further comprising:
an electronic component configured to receive an electrical signal from the sensor and output one or more values on a display, the one or more values correlating to the measured response.
